(19) United States
(12) Patent Application Publication
(10) Pub. No.: US 2003/0097098 A1
(43) Pub. Date: May 22, 2003

(54) Needle assembly and sheath and method of filling a drug delivery device

(76) Inventors: Gilad Lavi, Rishon Letzion (IL); Ehoud Carmel, Kiryat-Ono (IL); Izrail Tsals, Newton, PA (US)

Correspondence Address: ELAN HOLDINGS INC., 1300 GOULD DR., GAINESVILLE, GA, 30504, US

(21) Appl. No.: 09/813,537

(22) Filed: Mar. 21, 2001

Related U.S. Application Data
(60) Provisional application No. 60/192,885, filed on Mar. 29, 2000.

(51) Int. Cl. A61M 5/00

(52) U.S. Cl. 604/263

(57) ABSTRACT

A needle assembly has a needle holder that holds a needle and a sheath. The sheath has a needle compartment that accommodates the needle and any spilled drug flowing from the needle. The sheath and the needle holders are detachable. The sheath has an opening communicating with the needle compartment. A liquid barrier, which can comprise a hydrophobic membrane, is positioned in the first opening, blocking the first opening. The liquid barrier allows gas to escape through the first opening but prevent liquid from leaking through the first opening during drug filling stage. In use, the sheath is positioned over the needle of a drug delivery device. The drug delivery device is filled through a separate port until its drug reservoir is completely filled, which is accomplished by filling until the drug spills out through the needle. The sheath maintains the spilled drug within the needle compartment, while air bubbles escape through the liquid barrier.

### BACKGROUND

The present invention relates to drug delivery devices, and in particular to ambulatory, drug delivery devices. Prefilled drug delivery devices are desirable and are being adopted due to the significant advantages they provide over standard drug delivery devices, such as standard syringes that require filling by a health care worker immediately before administration. Benefits of such devices include user convenience, and decreased cost. Often, the user may administer the drug via the device. This can be done either within a physician's office, hospital, or at home. This results in an increased level of convenience for the user receiving the dosage and significant decrease in health care cost as a health care worker is not necessarily needed to administer the drug via the device. To prevent accidental needle sticks and contamination, the delivery needle associated with such a device typically may include a protective sheath that is removed immediately before use, or may be designed so that the needle is retracted into a protective casing before use.

Prefilled drug or medication delivery devices must be filled with liquid drug before they can be used. In most designs, the needle sheath shrouds and seals the needle-occupying compartment to prevent contamination. Sealing the needle compartment from ambient also traps air bubbles within its drug-holding compartment or drug reservoir. During priming or filling, air bubbles must be allowed to escape. Otherwise, back pressure will cause incomplete filling of the drug reservoir in the device or will prevent filling altogether. Therefore, to obtain full and accurate filling in present drug delivery device designs, the needle sheath must be detached during priming or filling, and then reattached after the priming is completed.

During priming, when the sheath is removed, or otherwise not attached, the needle can be exposed. Moreover, for accurate dosage, drug is typically filled until a small amount of drug is expelled through the needle. This ensures that 1) the drug reservoir is accurately filled and 2) that no air bubbles remain in the drug reservoir. The drug leakage, however, can be problematic as it can undesirably spill over to various components of the device, particularly if the needle end is exposed inside the device, e.g., retracted inside the device housing. In drug delivery devices that are designed to adhere to the skin surface during use, the leakage of drug during priming or filling is detrimental to the function of the device because the liquid drug may compromise the integrity of the adhesive.

Accordingly, there is a need to prevent the drug from spilling out of the needle.

Moreover, there is a need for enabling priming or filling such a device while the sheath remains attached to the device to enable air bubbles to escape through the sheath, but to prevent the drug from spilling out.

Moreover, there is a need for providing an improved needle sheath that enables a drug delivery device to be filled without exposing the needle to the external environment resulting in contamination of the needle and risk the safety and health of the user or health care worker.

There is yet a further need to provide an improved needle cover and sheath that is capable of allowing air to pass therethrough but prevents the escape of any drug, thus enabling the drug to remain in the device and be delivered accurately and completely to the user.

There is still a further need to provide an improved needle cover and sheath that captures any liquid drug flowing out of the device during filling or priming in a manner that will not compromise the integrity of the adhesive layer of the device.

The present invention addresses these needs.

### SUMMARY

The present invention is drawn to a needle assembly, a sheath thereof, and a method of filling or priming drug delivery device. The sheath is attachable to the needle assembly and allows fluid to flow out of the needle and capture any liquid expelled from the needle. Specifically, the sheath includes a liquid barrier that allows gas to escape but block passage of liquid therethrough.

Thus, according to one aspect of the invention, the needle assembly comprises a needle holder and a sheath. A further aspect of the present invention comprises a needle sheath for receiving at least a portion of a length of needle. The internal cavity of the sheath is shaped to funnel and hold a volume of liquid drug that flows out of the needle tip during priming or filling. Another aspect of the invention is the sheath with a liquid barrier, which can comprise a hydrophobic membrane or the like.

The needle holder is adapted to hold, or holds, a needle. The sheath has a needle compartment that can accommodate the needle, and has a first opening communicating with the needle compartment. The sheath is adapted to shroud at least a portion of the needle. The liquid barrier is positioned in the first opening and blocks the needle compartment so that the liquid barrier allows gas to escape through the first opening but prevent liquid from leaking through the first opening.

According to another aspect of the invention, the sheath can have a second opening, which communicates with the needle compartment, through which a portion of the needle holder is insertable into the sheath. The needle holder can have a projection dimensioned and configured to be inserted and frictionally seated into the second opening. The sheath can have a recess coaxially arranged with the second opening. The recess can seat the projection.

According to another aspect of the invention, the sheath can have a wall that closes one end of the compartment. The needle is inserted into the compartment by piercing through that wall.

According to another aspect of the invention, the sheath can have a central sheath portion housing the needle compartment and a flange portion extending laterally outwardly from the central sheath portion. The central portion is adapted to be inserted into a needle passage formed in a base of a drug delivery device while the flange portion abuts against a liner abutting the base so that the sheath is removable by removing the liner, which protects an adhesive layer.

According to another aspect of the invention, a support with an aperture is provided to support the hydrophobic member substantially concentrically positioned over the aperture. At least the support can be frictionally engaged to a surface of the sheath surrounding the first opening. Alternatively, the support can be seated in the first opening, holding the hydrophobic member in the first opening.

According to another aspect of the invention, the sheath for a needle has a central sheath portion adapted to be insertable through an opening of a drug delivery device and engage a portion of the needle, and a flange portion having a dimension larger than the central sheath portion and adapted to abut against an adhesive protector of the drug delivery device. Removing the adhesive protector can automatically remove the sheath from the needle in this embodiment. The central sheath portion and the flange portion can be monolithically formed of solid material, such as rubber.

Another aspect of the invention comprises a method of filling or priming a drug delivery device having a delivery needle, a drug reservoir, and a filling port. The method can comprise providing the afore-described sheath and engaging the sheath over the needle so that liquid expelled from the needle is confined within the needle compartment, flowing drug through the filling port to fill the drug reservoir or priming the drug reservoir until the drug flows out through the needle. The liquid barrier allows air bubbles to escape into ambient but traps the spilled drug within the needle compartment.

### BRIEF DESCRIPTION OF THE DRAWINGS

These and other features, aspects, and advantages of the present invention will become more apparent from the following description, appended claims, and accompanying exemplary embodiments shown in the drawings, which are briefly described below.

FIG. 1 is an exploded perspective view of a needle assembly having a sheath according to the present invention.

FIG. 2 is a cross-sectional view of the needle assembly of FIG. 1.

FIG. 3 is another embodiment of the sheath according to the present invention.

FIG. 4 is another embodiment of the sheath according to another aspect of the present invention.

### DETAILED DESCRIPTION

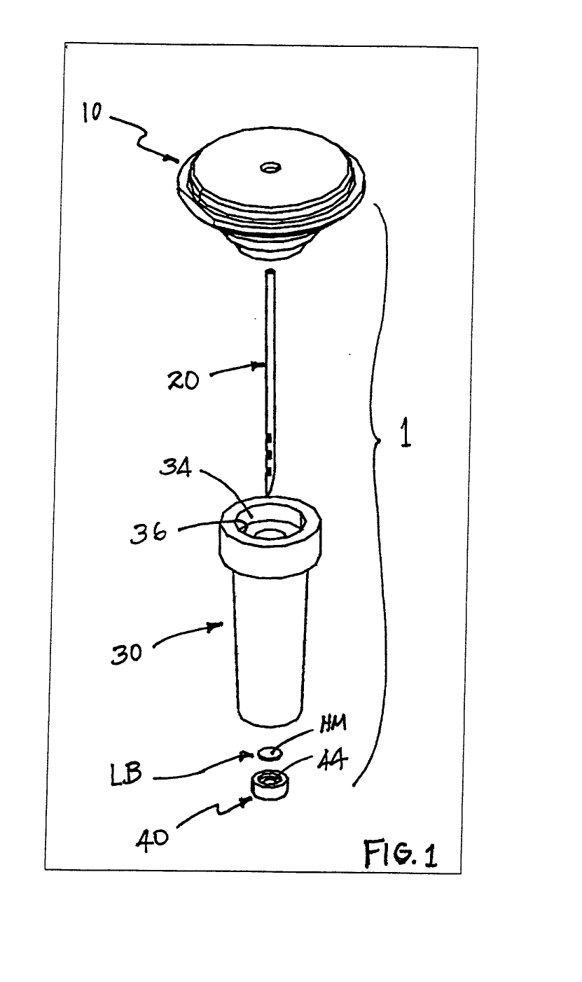

Three exemplary embodiments, as shown in FIGS. 1-4, serve to illustrate the present invention. In this regard, same or corresponding elements are labeled with the same or similar reference numerals. Moreover, although references are made below to directions in describing the structure, they are made relative to the drawings (as normally viewed) for convenience. The directions, such as left, right, upper, lower, etc., are not intended to be taken literally or limit the present invention in any form.

The needle assembly 1 comprises a needle holder 10, 10′ for holding a needle 20, and a needle sheath 30, 30′. The needle holder 10, 10′ can be, for instance, a conventional luer lock that can be detachably mounted to a syringe or injection device, or a hub or hub-like body that can be adapted for mounting to a drug delivery device, such as the type disclosed in U.S. Pat. No. 5,858,001. The needle holder 10, 10′ contemplated according to the present invention thus can be any suitable structure, whether it be adapted for a syringe or custom design, that can hold a needle 20 and connect to a drug delivery mechanism.

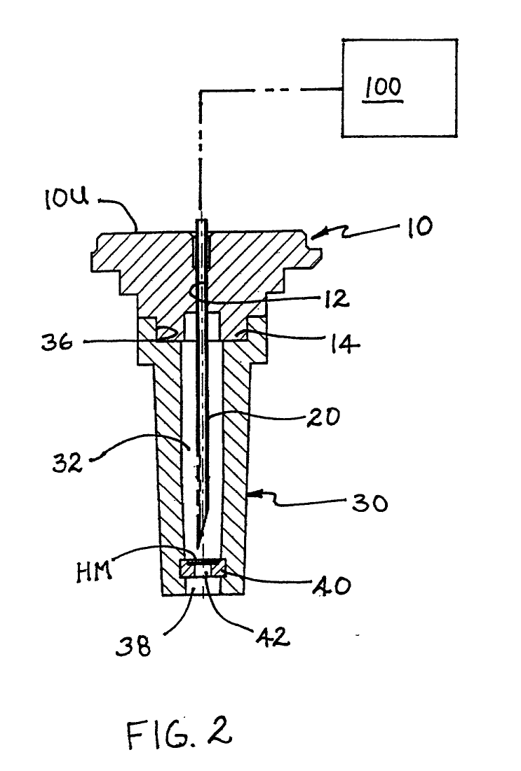

In the illustrated first and second embodiments, the needle holder 10, 10′ has a central axial through hole 12 for passage of the needle 20. Although these embodiments show the needle 20 extending completely through the needle holder 10, 10′, the needle 20 need not extend completely through. The needle holder 10, 10′ can be configured to work with a drug delivery mechanism 100, such as a syringe, to deliver the drug from the upper side lOU of the needle holder 10, 10′, as shown in FIG. 2, such as through a port or channel formed in the needle holder 10, 10′.

In the first embodiment, FIGS. 1-2, the needle holder 10 has a stepped portion that cooperates with the opening of the sheath 30 to sealingly, but detachably hold the sheath 30. Specifically, the stepped portion is formed, for example, by a smaller dimensioned annular projection 14 extending axially downwardly. The projection 14 is dimensioned and configured to be inserted and frictionally seated into an opening of the sheath 30 or a twist type mounting mechanism, e.g., bayonet mount, threaded connection.

In the first and second embodiments, the sheath 30, 30′ has a hollow and elongated portion, and has a needle compartment 32 for receiving the needle 20. The needle compartment is configured to funnel and hold any liquid expelled from the needle 20. In the first embodiment, the sheath 30 has an opening 34 with a recess 36 at one end. The opening 34 communicates with the needle compartment 32. The recess 36 is dimensioned and configured to sealingly receive the projection 14.

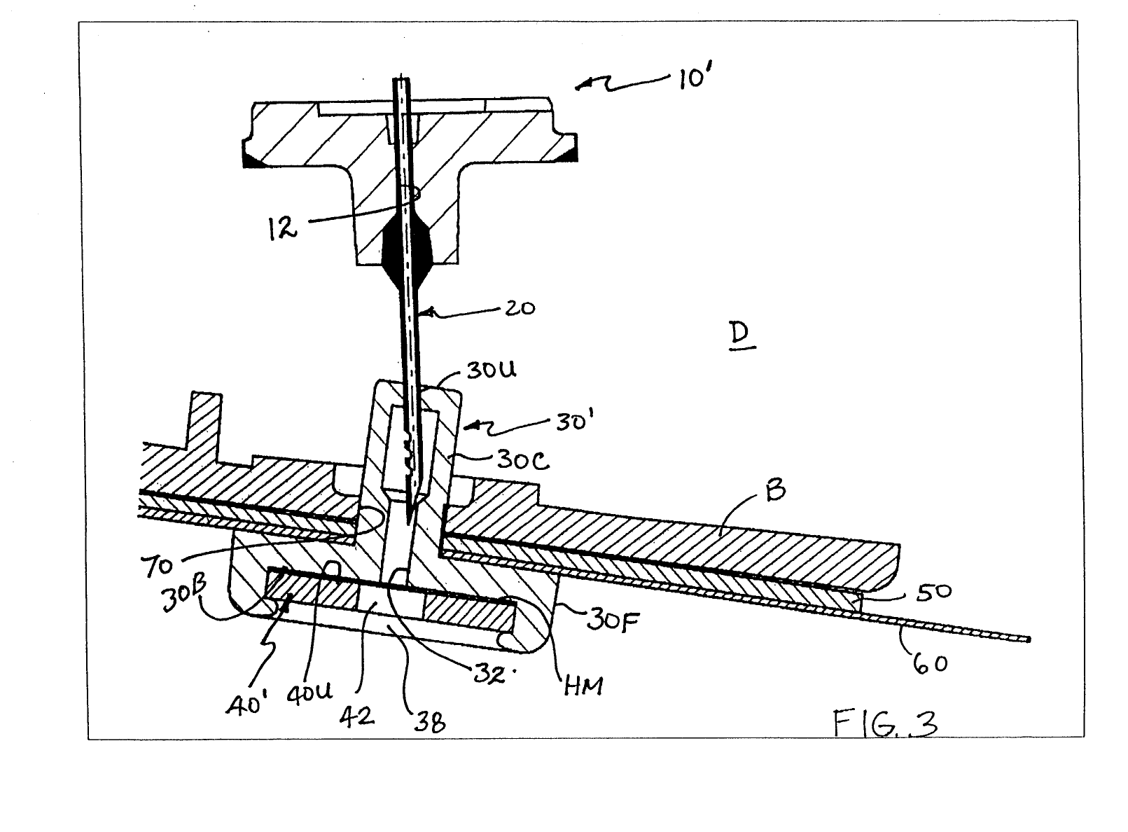

The second embodiment, FIG. 3, illustrates a needle assembly that is particularly suitable for a drug delivery of the type disclosed in U.S. Pat. No. 5,858,001, the disclosure of which is incorporated herein by reference. In this embodiment, the needle holder 10′ is substantially similar to that of the first embodiment, except that the sheath 30′ has a different configuration. In the second embodiment, the needle holder 10′ does not matingly and sealingly engage the sheath 30′, as occurs in the first embodiment. Instead, the needle 20 itself punctures through a closed upper end or upper wall 30U of the sheath 30′.

The sheath 30, 30′ can be formed of any suitable material, but preferably is made of rubber, such as a bromobutyl rubber, or any other suitable commercial material.

According to one aspect of the present invention, the sheath 30, 30′ has a liquid barrier LB that lets gas, e.g., air bubbles, escape through the sheath 30, 30′, while blocking passage of drug therethrough. This is particularly advantageous in a drug delivery device, where the drug is filled through a separate drug filling port. In particular, during filling or priming, the drug can be accurately filled by allowing any trapped air bubbles to escape through the needle and through the sheath 30, 30′ but confine any liquid expelled from the needle within the needle compartment 32. As gas, e.g., air bubbles, can escape during the filling stage, no backpressure is developed to enable an accurate dosage.

According to the present invention, the liquid barrier LB comprises a hydrophobic disc or membrane HM positioned inside the sheath 30, 30′. The hydrophobic membrane HM is a material, such as a polymer with fine pores, which allows passage of air or gas thereacross or therethrough but repel liquid. Examples of suitable hydrophobic membrane HM are SUPOR, available from PALL SPECIALTY MATERIAL, IV FILTER available from WHATMAN, and IMMOBILON from MILLIPORE.

To accommodate the hydrophobic membrane HIM, the sheath 30, 30′ can have an opening or aperture 38 that communicates with ambient and the needle compartment 32. The hydrophobic membrane 1M sealingly covers or blocks the opening 38. For instance, the opening 38 can be formed at a lower end of the sheath, below the needle 20, as illustrated in FIGS. 1-3. The hydrophobic membrane HM should be positioned away from the needle point so that it does not become punctured. If the sheath is made of rubber or relatively flexible or soft material, it is preferable to support the hydrophobic membrane HM with a relatively rigid support 40, 40′, which can be formed of a plastic, for example. Alternatively, the sheath could have a closed bottom with an opening. The sheath can be dimensioned to sealingly seat the hydrophobic membrane HM over the opening.

The first and second embodiments both have the support 40, 40′, configured substantially cylindrically, but can have any suitable configuration. The support 40, 40′ has a central aperture 42, which provides a pathway for gas to escape through the hydrophobic membrane HM. In the first embodiment, the support 40 has a recess 44 for sealingly seating the hydrophobic membrane HM. In the first embodiment, the inner surface 32S of the sheath surrounding the opening 38, or the needle compartment 32, frictionally and sealingly engages the outer periphery of the support 40 to immobilize the support. That is, the support 40 with the hydrophobic membrane HM is inserted or pushed up into the opening 38 or the needle compartment, and frictionally secured thereto.

In the second embodiment, the opening 38 is collinear with the needle compartment, but has a larger dimension or diameter (in the case of a cylindrical support). The hydrophobic membrane HM is positioned to block the lower end of the needle compartment 32, and is sealingly sandwiched between an upper side 40U of the support 40′ and a bottom surface 30B of the opening 38. The lower portion of the sheath 30′ surrounding the opening 38 is dimensioned larger, forming a flange portion 30F. The opening 38 can be formed in the flange portion 30F. The flange portion 30F also includes a lip 30L that extends radially inwardly at the entrance of the opening. The support 40′ is forced into the opening 38. The lip 30L helps to maintain the support 40′ securely confined in the opening 38.

In the second embodiment, the sheath 30′ is configured to protect an adhesion layer 50 typically used for attaching a drug delivery device to a drug delivery site. That is, during priming or filling, overfilling the drug reservoir, for purposes of completely filling the same removing any trapped air bubbles, results in spillage. The drug can undesirably wet the adhesion layer and make it less effective or completely ineffective. The sheath 30′ funnels and holds excess liquid drug that flows out of the needle during priming or filling. This design prevents spillage and protects the adhesion layer 50 from exposure to any liquid drug.

In the second embodiment, the liner 60 protecting the adhesion layer 50 can be used to remove the sheath 30′. In this respect, the sheath 30′ has a central sheath portion 30C that extends through a needle opening 70 formed through a base portion B of the drug deliver device D, and shrouds the needle 20. The flange portion 30F, which is dimensioned larger than the central sheath portion 30C, abuts against the underside of the base portion B. Pulling the liner 60 off the base B forces the sheath 30′ to move outwardly off the base, removing the sheath 3 0′ from the drug delivery device D.

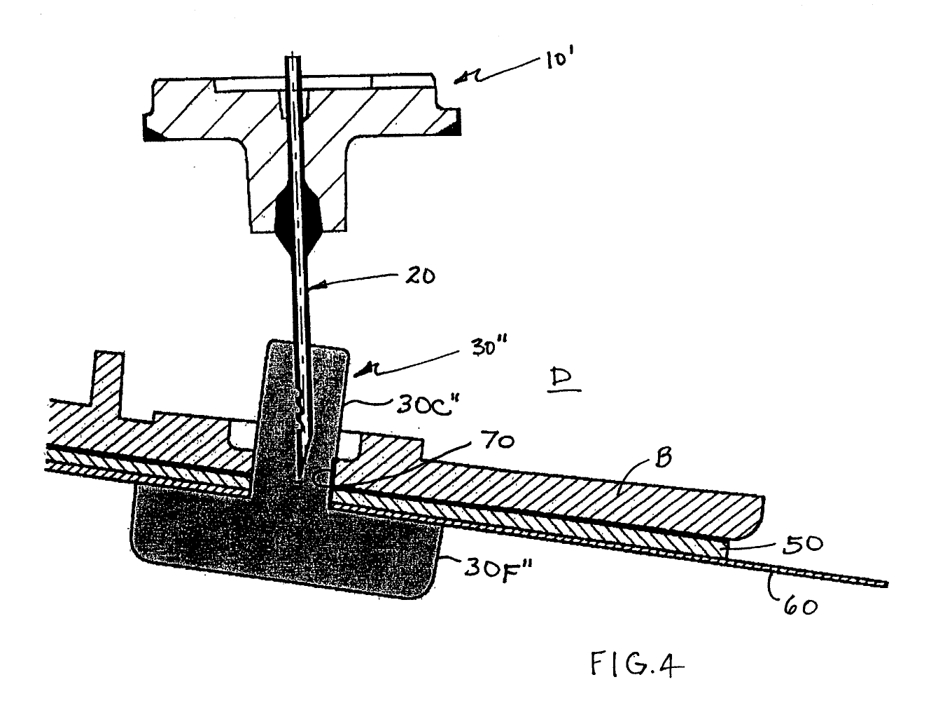

The third embodiment, FIG. 4, is similar to the second embodiment, FIG. 3, except that the sheath 30″ is solid, with no needle accommodating compartment. The sheath 30″ has a central sheath portion 30C″ that extends through the needle opening 70 formed through a base portion B of the drug deliver device D and shrouds the needle 20. The flange portion 30F″, which is dimensioned larger than the central sheath portion 30C″, abuts against the underside of the base portion B. Pulling the liner 60 off the base B forces the sheath 30″ to move outwardly off the base, removing the sheath 30″ from the drug delivery device D.

In the third embodiment, the needle pierces through the central sheath portion 30″ and blocks the needle, preventing the drug from spilling out of the needle. The sheath 30″ also can be made of rubber, preferably a solid rubber or the like.

According to a method of filling or priming a drug delivery device, which typically has a delivery needle, a drug reservoir, and a filling port, according to the present invention, the sheath is placed over the needle before filling take place. If the drug deliver device is provided with a prefilled cartridge, the device is primed while the sheath is placed over the needle. The drug reservoir is filled or primed until the drug spills out through the needle to remove air bubbles. The liquid barrier allows air bubbles to escape into ambient, but traps the spilled drug within the needle compartment.

Given the disclosure of the present invention, one versed in the art would appreciate that there may be other embodiments and modifications within the scope and spirit of the present invention. Accordingly, all modifications attainable by one versed in the art from the present disclosure within the scope and spirit of the present invention are to be included as further embodiments of the present invention. The scope of the present invention accordingly is to be defined as set forth in the appended claims.

We claim:

1. A needle assembly comprising:
a needle holder adapted to hold a needle; and
a sheath having a needle compartment for accommodating the needle, the sheath being adapted to shroud at least a portion of the needle, the sheath allowing fluid to flow out of the needle and capture any liquid expelled from the needle within the sheath.

2. A needle assembly according to claim 1, wherein the sheath has a first opening communicating with the needle compartment, and further comprising a liquid barrier positioned in the first opening so that the liquid barrier allows gas to escape through the first opening, but prevent liquid from leaking through the first opening.

3. A needle assembly according to claim 2, wherein the liquid barrier comprises a hydrophobic membrane.

4. A needle assembly according to claim 1, wherein the sheath has a second opening through which a portion of the needle holder is insertable into the sheath, the second opening communicating with the needle compartment.

5. A needle assembly according to claim 4, wherein the needle holder has a projection dimensioned and configured to be inserted and frictionally seated into the second opening.

6. A needle assembly according to claim 5, wherein the sheath has a recess coaxially arranged with the second opening, the recess seating the projection.

7. A needle assembly according to claim 1, wherein the sheath has a wall that closes one end of the compartment, wherein the needle is inserted into the compartment by piercing through the wall.

8. A needle assembly according to claim 7, wherein the sheath has a central sheath portion housing the needle compartment and a flange portion extending laterally outwardly from the central sheath portion, the central portion being adapted to be inserted into a needle passage formed in a base of a drug delivery device while the flange portion abuts against a liner abutting the base so that the sheath is removable by removing the liner, which protects an adhesive layer.

9. A needle assembly according to claim 3, further including a support with an aperture, the hydrophobic member resting on the support with the hydrophobic member substantially concentrically positioned over the aperture.

10. A needle assembly according to claim 9, wherein at least the support is frictionally engaged to a surface of the sheath surrounding the first opening.

11. A needle assembly according to claim 9, wherein the support is seated in the first opening, holding the hydrophobic member in the first opening.

12. A sheath for a needle, comprising:
a needle compartment for shrouding over at least a portion of the needle, the needle compartment allowing fluid to flow out of the needle and capture any liquid expelled from the needle.

13. A sheath according to claim 12, further including a first opening communicating with the needle compartment, and a liquid barrier positioned in the first opening so that the liquid barrier allows gas to escape through the first opening but prevent liquid from leaking through the first opening.

14. A sheath according to claim 12, wherein the needle compartment is dimensioned to funnel and hold liquid drug expelled from the needle.

15. A sheath according to claim 13, wherein the liquid barrier comprises a hydrophobic membrane.

16. A sheath according to claim 13, further including a second opening adapted for receiving and seating a needle holder, the second opening communicating with the needle compartment.

17. A sheath assembly according to claim 16, further including a recess coaxially arranged with the second opening and adapted to seat a portion of the needle holder.

18. A sheath according to claim 13, wherein the sheath has a wall that closes one end of the needle compartment, wherein the needle is inserted into the compartment by piercing through the wall.

19. A sheath according to claim 16, wherein the sheath has a central sheath portion housing the needle compartment and a flange portion extending laterally outwardly from the central sheath portion, the central portion being adapted to be inserted into a needle passage formed in a base of a drug delivery device while the flange portion abuts against a liner abutting the base so that the sheath is removable by removing the liner, which protects an adhesive layer.

20. A sheath according to claim 15, further including a support with an aperture, the hydrophobic member resting on the support with the hydrophobic member substantially concentrically positioned over the aperture.

21. A sheath according to claim 20, wherein at least the support is frictionally engaged to a surface of the sheath surrounding the first opening.

22. A sheath according to claim 21, wherein the support is seated in the first opening, holding the hydrophobic member in the first opening.

23. A method of filling or priming a drug delivery device having a delivery needle, a drug reservoir, and a filling port, comprising:
providing a sheath having a needle. compartment for accommodating the needle, a first opening communicating with the needle compartment, and a liquid barrier positioned in the first opening so that the liquid barrier allows gas to escape through the first opening but prevent liquid from leaking through the first opening;
engaging the sheath over the needle so that liquid expelled from the needle is confined within the needle compartment;
flowing drug through the filling port to fill the drug reservoir or priming the drug reservoir until the drug flows out through the needle,
wherein the liquid barrier allows air bubbles to escape into ambient, but trapping the spilled drug within the needle compartment.

24. A method according to claim 23, wherein the liquid barrier is a hydrophobic membrane.

25. A sheath for a needle, comprising:
a central sheath portion adapted to be insertable through an opening of a drug delivery device and engage a portion of the needle; and
a flange portion having a dimension larger than the central sheath portion and adapted to abut against an adhesive protector of the drug delivery device.

26. A sheath according to claim 25, wherein the central sheath portion and the flange portion are monolithically formed of solid material.
